(19) United States
(12) Patent Application Publication
(10) Pub. No.: US 2026/0263462 A1
(43) Pub. Date: Sep. 10, 2026

(54) BLU-945 IN COMBINATION WITH OSIMERTINIB FOR THE TREATMENT OF NON-SMALL-CELL LUNG CANCER

(71) Applicant: Blueprint Medicines Corporation, Cambridge, MA (US)

(72) Inventors: Chiara Conti, Cambridge, MA (US); Alena Zalutskaya, Cambridge, MA (US)

(21) Appl. No.: 18/849,277

(22) PCT Filed: Mar. 22, 2023
(86) PCT No.: PCT/US2023/015849

§ 371 Date: Sep. 20, 2024

Related U.S. Application Data
(60) Provisional application No. 63/322,371, filed on Mar. 22, 2022.
(60) Provisional application No. 63/417,489, filed on Oct. 19, 2022.
(60) Provisional application No. 63/421,516, filed on Nov. 1, 2022.

(51) Int. Cl. A61K 31/506; A61P 35/04

(52) U.S. Cl. CPC A61K 31/506; A61P 35/04

(57) ABSTRACT

The present disclosure provides improved methods of treating non-small cell lung cancer using N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1- yl)isoquinolin-3-amine as represented by the structure below: or a pharmaceutically acceptable salt thereof.

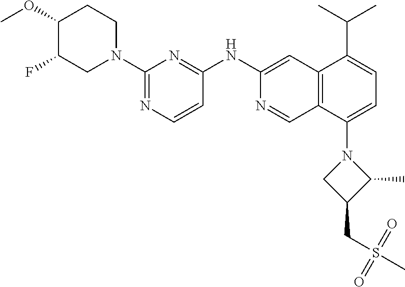

### CROSS REFERENCE TO RELATED APPLICATIONS

This application claims the benefit of U.S. Provisional Application Ser. No. 63/322,371, filed Mar. 22, 2022, U.S. Provisional Application Ser. No. 63/417,489, filed Oct. 19, 2022 and U.S. Provisional Application Ser. No. 63/421,516, filed Nov. 1, 2022. The entire teachings of each of the foregoing applications are incorporated herein by reference.

### BACKGROUND

Epidermal growth factor receptor mutations (EGFRm), the most frequent oncogenic drivers of non-small cell lung cancer (NSCLC), are found in ~17% of Caucasian and up to 50% of Asian patients (Zhang Y L et al. Oncotarget. 2016; 7:78985-78993, Shi Y et al. J Thorac Oncol. 2014; 9:154-162) with the most common being exon 19 deletion (ex19del) and L858R (Leonetti A et al. Br J Cancer. 2019; 121:725-737).

Use of tyrosine kinase inhibitors (TKIs) such as osimertinib has prolonged survival, but resistance eventually develops via multiple on- and off target mutations, often simultaneously found in distinct clones within an individual patient. The most frequent resistance mutations to emerge on therapy are EGFR T790M (after first [1G]-, and second [2G]-generation TKIs) and EGFR C797S (after third [3G]-generation TKIs) (Niederst M J et al. Clin Cancer Res. 2015; 21:3924-3933), there are currently no approved targeted therapies after progression on 3G TKIs, including those that target both C797S and T790M (Park S et al. Cancer Res Treat. 2020; 52:1288-1290).

An additional advancement to the treatment paradigm of EGFRm NSCLC is preventing emergence of multiple resistance mutations in the treatment naïve setting (Planchard D et al. ESMO Open. 2021; 6:100271, Cho B C et al. Future Oncol. 2022; 18:639-647) and several combination studies are currently underway to address this heterogeneity. (Yu H et al. Ann Oncol. 2021; 32 (suppl 5):S978-979; Miura S et al. J Thorac Oncol. 2021; 16 (suppl):S890-891; Bauml J et al. J Clin Oncol. 2021; 39 (suppl 15):9006).

International Application Publication No. WO 2021/133809, the entire teachings of which are incorporated herein by reference, discloses N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine, shown below (also referred herein as “BLU-945” or N-{2-[(3S,4R)-3-fluoro-4-methoxypiperidin-1-yl]pyrimidin-4-yl}-8-[(2R,3S)-3-(methanesulfonylmethyl)-2-methylazetidin-1-yl]-5-(propan-2-yl)isoquinolin-3-amine).

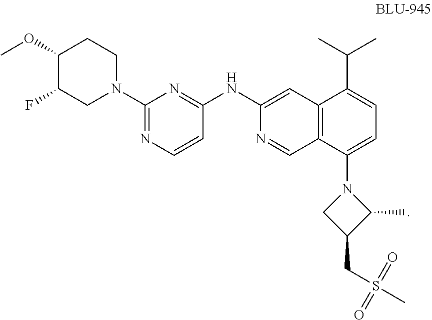

BLU-945 is an investigational, oral next-generation EGFR TKI designed to target EGFR T790M-carrying clones (regardless of driver mutation), including those with C797S (Schalm S S et al. Annals of Oncology. 2020; 31:S839). In addition, it inhibits the L858R regardless of the presence of T790M and C797S mutations at clinically relevant exposures. BLU-945 targets common EGFR activating (e.g., ex19del and L858R) and T790M and C797X resistance mutations, while being selective against EGFR wild-type (WT).

The high selectivity of BLU-945 allows mutational targeting without inhibition of wild type (WT) EGFR, which may enable combination with other TKIs that can address multiple mechanisms of resistance without added toxicity. BLU-945 has demonstrated in vivo antitumor monotherapy activity in treatment-naïve EGFRm xenograft and osimertinib-resistant EGFRm models with C797S (Miura S et al. J Thorac Oncol. 2021; 16 (suppl):S890-891), with and without T790M (Lim S M et al. Cancer Res. 2021; 81:1467-1467, Tavera L et al. AACR 2022 presentation; poster #3328).

In osimertinib resistant tumor models, BLU-945 has demonstrated enhanced antitumor activity in combination with complementary EGFR TKIs such as osimertinib (Tavera L et al. Lung Cancer. 2022; 165 (suppl 1):S78) and the next-generation EGFR TKI BLU-701 (Tavera L et al. AACR 2022 presentation; poster #3328).

### SUMMARY

Disclosed herein are improved methods for treating a subject with a non-small cell lung cancer with N-(2-((3 S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine or a pharmaceutically acceptable salt thereof. The disclosure provides dosing regimens of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine for the treatment of non-small cell lung cancer. More specifically, the disclosure provides methods for treating a subject with a non-small cell lung cancer (NSCLC) by administering N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine orally in an amount of 200 mg to 1000 mg daily. Alternatively, N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine is administered orally in an amount of 100 mg to 1000 mg daily. An object of this disclosure is to provide new methods of treating a subject with a non-small cell lung cancer with a safe and effective daily dose.

In one aspect, the present disclosure provides a method of treating a subject with NSCLC, comprising administering to the subject in need thereof orally an effective amount of N-(2-((3 S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine or a pharmaceutically acceptable salt thereof and co-administering an effective amount of osimertinib.

In some embodiments, an amount of 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine represented by the structure below:

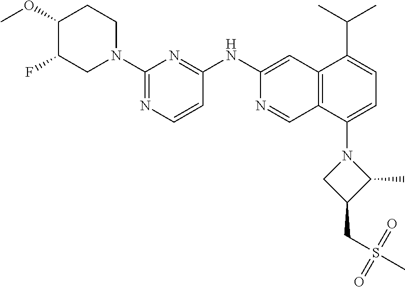

or a pharmaceutically acceptable salt thereof in an amount equivalent to 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine is administered daily. Alternatively, N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine is administered orally in an amount of 100 mg to 1000 mg daily, or a pharmaceutically acceptable salt thereof is administered orally in an amount equivalent to 100 mg to 1000 mg daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

The present disclosure also provides the oral use of 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine daily for the preparation of a medicament for the treatment of NSCLC. Alternatively, 100 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 100 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine daily is used. In some embodiments, the medicament is used in combination with an effective amount of osimertinib.

In another aspect, 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof is provided herein in an amount equivalent to 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine for oral and daily use in treating a subject with NSCLC, optionally in combination with an effective amount of osimertinib. Alternatively, 100 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof is provided herein in an amount equivalent to 100 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine for oral and daily use in treating a subject with NSCLC, optionally in combination with an effective amount of osimertinib.

### BRIEF DESCRIPTION OF THE DRAWINGS

FIG. 1 shows the study design of ongoing Phase 1/2, open-label, first-in-human study (NCT04862780). ctDNA, circulating tumor DNA; DUSP6, dual specificity phosphatase 6; ECG, electrocardiogram; ECOG PS, Eastern Cooperative Oncology Group performance status; EGFRm, mutant epidermal growth factor receptor gene; MTD, maximum tolerated dose; NSCLC, non-small cell lung cancer; ORR, overall response rate; OS, overall survival; QD, once daily; RP2D, recommended phase 2 dose; SPRY4, sprouty RTK signaling antagonist 4; TKI, tyrosine kinase inhibitors.

^{a}Based on Bayesian Optimal Interval (BOIN) escalation design; ^{b}BID dosing will also be evaluated; ^{c}Part 1B and Phase 2 have not been initiated and are dependent on Part 1A results.

FIG. 2A shows reduced EGFR T790M ctDNA levels with BLU-945 treatment*.

FIG. 2B shows reduced EGFR C797S ctDNA levels with BLU-945 treatment*.

*Patient had two different DNA mutations in C797S. Note: reductions in individual variant allele fractions as shown; therefore, patients with multiple mutations may be represented on both plots. All T790M and C797S allele fractions with available baseline and C1D15 data are shown. Increases of greater than 100% were truncated at 100%.

FIG. 3 shows dose-dependent tumor shrinkage with BLU-945 treatment^{a,b}. ^{a}Patients with measurable target lesions at baseline with post-baseline scans; ^{b}Data cut off, 9 Mar. 2022.

FIG. 4 shows longitudinal ctDNA profiling of two patients.

FIG. 5 shows BLU-945 monotherapy dose escalation data, demonstrating reduced EGFR C797S ctDNA levels (left panel) and tumor shrinkage (right panel) with BLU-945 treatment. BID=twice daily; C1D1=cycle 1 day 1; CID15=cycle 1 day 15; PD=progressive disease; PR=partial response; SD=stable disease; VAF=variant allele fraction.

FIG. 6 is a bar graph showing changes in EGFR Variant Allele Fraction in ctDNA from Baseline to CID15 of BLU-945 Monotherapy. Abbreviations: BID=twice daily; EGFR=epidermal growth factor receptor; FMI=functional mathematical index; QD=once daily; VAF=variant allele fraction. Changes in EGFR-T790M and EGFR-C797S variant allele fractions (VAFs) at CID15 calculated using each patient's pre-treatment baseline (C1D1) VAF. Detected baseline (C1D1) EGFR mutational profiles (using FMI F1LCDx assay) detailed below each plot. *indicates patient had two distinct DNA molecules detected giving rise to the same protein alteration. In such cases, changes in the VAF of each respective DNA molecule is plotted separately, so multiple bars on each plot may belong to the same patient. \hat{ } indicates the patient's C2D1 specimen was used for the analysis in place of CID15, in addition, the LR mutation was not initially detected at baseline (CID1).

FIG. 7 is a bar graph showing that sum of diameter of target lesion by initial dose level, BLU-945 Monotherapy ≥400 mg Per Day. Abbreviations: BID=twice daily; QD=once daily. The maximum percentage reduction is calculated before the disease progression.

FIG. 8 is a bar graph showing changes in EGFR Variant Allele Fraction in ctDNA from Baseline to CID15 of BLU-945 and Osimertinib in Combination. Abbreviations: BID=twice daily; EGFR=epidermal growth factor receptor; FMI=functional mathematical index; osi=osimertinib; QD=once daily; VAF=variant allele fraction. Changes in EGFR variant allele fractions (VAFs) at CID15 are calculated using each patient's pre-treatment baseline (C1D1) VAF. Detected baseline (C1D1) EGFR mutational profiles (using FMI F1LCDx assay) detailed in each plot. * indicates patient had two distinct DNA molecules detected giving rise to the same protein alteration. In such cases, changes in the VAF of each respective DNA molecule is plotted separately, so multiple bars on each plot may belong to the same patient.

FIG. 9 is a bar graph showing the sum of diameter of target lesion by initial dose level, BLU-945 in Combination with Osimertinib 80 mg QD, all doses. Abbreviations: BID=twice daily; osi=osimertinib; QD=once daily. The maximum percentage reduction is calculated before the disease progression.

### DETAILED DESCRIPTION

### Methods of Treatment

Despite significant advances, including three generations of EGFR tyrosine kinase inhibitors (TKIs), patients with advanced EGFR mutant NSCLC will inevitably progress, with a median PFS of 18.8 months overall with osimertinib treatment in the front line, and even shorter in poor risk subgroups, like patients with EGFR L858R (14.4 months), detectable ctDNA at baseline (11.1 months) or detectable ctDNA after 3 weeks of therapy (9.5 months) (Zhou C, Imamura F, Cheng Y, Okamoto I, Cho B C, Lin M C, et al. Early clearance of plasma EGFR mutations as a predictor of response to osimertinib and comparator EGFR-TKIs in the FLAURA trial. J Clin Oncol. 2019; 37(15 suppl) ASCO Annual Meeting Abstract 9020). Though treatment after osimertinib is an important area of unmet medical need, patients very often develop multiple on- and off-target escape mechanisms, which will likely require both EGFR and non-EGFR pathway inhibition to address.

On the other hand, deepening and extending responses in the front-line setting, before complex heterogeneous resistance mechanisms occur, is an important opportunity to improve outcomes for patients. Several strategies involving combinations designed to delay or prevent resistance and extend front line responses are being investigated, including osimertinib in combination with platinum-based chemotherapy and amivantamab and lazertinib in all EGFR mutated patients, as well as poor risk subsets, such as ramucirumab and erlotinib in L858R patients and osimertinib and chemotherapy in patients with detectable ctDNA at 3 weeks. Each of these strategies involve IV therapies and combinations with significant toxicity.

Although the combination of two EGFR TKIs has the potential to improve outcomes by more potently inhibiting primary EGFR driver mutations and covering a broader range of EGFR resistance mutations, prior combinations have been limited by the increase in wild type (WT) EGFR adverse events (AEs), which is dose limiting.

BLU-945 is an investigational, next generation EGFR TKI which covers the common on target resistance mutations, T790M and C797S, and is highly selective against WT EGFR, resulting in minimal WT EGFR AEs. Preclinical data has shown antitumor activity on osimertinib resistant models, and enhanced activity in combination with osimertinib in treatment naïve L858R models, with improved durability of response and survival compared to BLU-945 or osimertinib alone. (See “BLU-945 or BLU-701 as single agents versus their combination with osimertinib in EGFR L858R-driven tumor model”, Tavera-Mendoza, LE, AACR-NCI-EORTC 2022, Barcelona, Spain, Oct. 26-28, 2022) As of 6 Jan. 2023, 108 patients have been treated with BLU-945 monotherapy and 25 patients have been treated with BLU-945 in combination with osimertinib. In a heavily pre-treated population, BLU-945 monotherapy has demonstrated a generally well tolerated and differentiated safety profile with few discontinuations for related AEs and minimal WT EGFR AEs, as well as evidence of robust on target activity on EGFR with reductions and clearance of EGFR resistance mutations and confirmed responses in a genomically heterogeneous population with multiple oncogene disease drivers. For example, 400 mg QD, 500 mg QD, 200 mg BID, and 250 mg BID BLU-945 dosages can be used in the treatment of NSCLC, as 600 mg QD and 300 mg BID of BLU-945 were not tolerated by patients.

BLU-945 in combination with osimertinib has been generally well tolerated in dose escalation, with few discontinuations for related AEs and consistent with the safety profile of both agents, with no evidence of an increased WT AE frequency over osimertinib nor new safety signals from the combination. Responses, pending confirmation, have occurred in patients that previously progressed on osimertinib, at lower doses of BLU-945 than observed in monotherapy.

Given the need to develop new therapy regimens to improve outcomes in front line NSCLC, the preclinical activity of BLU-945 and osimertinib in extending anti-tumor responses in front line models, the tolerability of BLU-945 monotherapy and BLU-945 in combination with osimertinib and promising activity in late line patients, the available data supports an acceptable benefit/risk ratio for continuing BLU-945 clinical development, including the addition of BLU-945 to the standard of care osimertinib in front line patients with advanced EGFR mutant NSCLC.

The present disclosure provides a method of treating a subject with a non-small cell lung cancer (NSCLC), comprising administering to the subject in need thereof orally an amount of 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine, or a pharmaceutical composition disclosed comprising the same daily.

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine disclosed herein can be useful in the form of a free base or as a salt. Representative salts include the hydrobromide, hydrochloride, sulfate, bisulfate, phosphate, nitrate, acetate, valerate, oleate, palmitate, stearate, laurate, benzoate, lactate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

In some embodiments, in the methods of treatment disclosed herein, N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine is administered as the free base.

In some embodiments, in the methods of treatment disclosed herein, a pharmaceutically acceptable salt of N-(2-((3S,4R)-3-fluoro-4-methoxypiperidin-1-yl)pyrimidin-4-yl)-5 -isopropyl-8-((2R,3 S)-2-methyl-3-((methylsulfonyl)methyl)azetidin-1-yl)isoquinolin-3-amine is administered.

When the stereochemical configuration at a chiral center in N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

In some embodiments, the non-small cell lung cancer in the subject in need thereof has metastasized (e.g. to the brain).

In some embodiments, NSCLC is locally advanced or metastatic NSCLC, NSCLC adenocarcinoma, NSCLC with squamous histology and NSCLC with non-squamous histology.

In some embodiments, the non-small cell lung cancer is characterized by epidermal growth factor receptor (EGFR) L858R mutation. In some embodiments, the non-small cell lung cancer is characterized by epidermal growth factor receptor (EGFR) exon 19 deletion mutation. In some embodiments, NSCLC is characterized by EGFR L858R/T790M mutation or EGFR exon 19 deletion/T790M mutation. In some embodiments, NSCLC is characterized by EGFR L858R/C797X mutation or EGFR exon 19 deletion/C797X mutation (where “X” can be an “S” or a “G” or an “N” or a “Y” or a “T” or a “D”). In some embodiments, NSCLC is characterized by EGFR L858R/T790M/C797X mutation or EGFR exon 19 deletion/T790M/C797X mutation. In one specific embodiment, the C797X mutation is C797S mutation.

In another embodiment, the NSCLC associated with or modulated by mutant EGFR that is characterized by an EGFR genotype selected from genotypes 1-76 according the Table below (del18=Exon 18 deletion, specifically, e.g., del E709_T710 insD; and del19=Exon 19 deletion, specifically, e.g., delE746_A750 (most common), delE746_S752insV, del747_A750insP, delL747_P753insS, and delS752_1759; ex20ins—Exon 20 insertion, specifically, e.g., D761-E762insX, A763-Y764insX, Y764-V765insX, V765-M766insX, A767-S768insX, S768-D769insX, V769-D770insX, N771-P772insX, P772-H773insX, H773-V774insX, and V774-C775insX):

### EGFR Genotype

| | |
|---|---|
| 1 | EGFR del19 |
| 2 | EGFR del19 T790M |
| 3 | EGFR del19 C797S |
| 4 | EGFR del19 C797X (C797G or C797N) |
| 5 | EGFR del19 T790M C797S |
| 6 | EGFR del19 T790M C797S Q791P |
| 7 | EGFR del19 T790M (C797G or C797N) |
| 8 | EGFR del19 L792X (L792F, L792H or L792Y) |
| 9 | EGFR del19 T790M L792X (L792F, L792H, or L792Y) |
| 10 | EGFR del19 G796R (G796S) |
| 11 | EGFR del19 T790M G796R (G796S) C797S L792X (L792F, |
| | L792H or L792Y) |
| 12 | EGFR del19 L792R (L792V or L792P) |
| 13 | EGFR del19 L718Q (L718V) |
| 14 | EGFR del19 T790M L718Q (L718V) L792X (L792F, L792H |
| | or L792Y) |
| 15 | EGFR del19 T790M G796R (G796S) |
| 16 | EGFR del19 T790M L792R (L792V or L792P) |
| 17 | EGFR del19 T790M L718Q (L718V) |
| 18 | EGFR del19 T790M C797S L718Q (L718V) |
| 19 | EGFR del19 G724S |
| 20 | EGFR del19 T790M G724S |
| 21 | EGFR del19 S768I (SV768IL) |
| 22 | EGFR del19 T790M S768I (SV768IL) |
| 23 | EGFR del19 T790M C797S/G L792X (L792F, L792H, |
| | L792R, or L792Y) |
| 24 | EGFR del19 V834L |
| 25 | EGFR del19 T790M V834L |
| 27 | EGFR del19 T790M L792X (L792F, L792H, L792R, or L792Y) |
| 28 | EGFR del19 C797S L718Q (L718V) |
| 29 | EGFR del19 L718Q (L718V) A750P |
| 30 | EGFR del19 T790M L718Q (L718V) A750P L792V G796R |
| 31 | EGFR L858R |
| 32 | EGFR L858R T790M |
| 33 | EGFR L858R C797S |
| 34 | EGFR L858R C797X (C797G or C797N) |
| 35 | EGFR L858R T790M C797S |
| 36 | EGFR L858R T790M C797S Q791P |
| 37 | EGFR L858R T790M C797X (C797G or C797N) |
| 38 | EGFR L858R L792X (L792F, L792H or L792Y) |
| 39 | EGFR L858R T790M L792X (L792F, L792H or L792Y) |
| 40 | EGFR L858R G796R (G796S) |
| 41 | EGFR L858R T790M G796R (G796S) C797S L792X (L792F, |
| | L792H or L792Y) |
| 42 | EGFR L858R L792R (L792V or L792P) |
| 43 | EGFR L858R L718Q (L718V) |
| 44 | EGFR L858R T790M G796R (G796S) |
| 45 | EGFR L858R T790M L792R (L792V or L792P) |
| 46 | EGFR L858R T790M L718Q (L718V) |
| 47 | EGFR L858R T790M C797S L718Q (L718V) |
| 48 | EGFR L858R T790M L718Q (L718V) L792X (L792F, |
| | L792H or L792Y) |
| 49 | EGFR L858R G724S |
| 50 | EGFR L858R T790M G724S |
| 51 | EGFR L858R S768I (SV768IL) |
| 52 | EGFR L858R T790M S768I (SV768IL) |
| 53 | EGFR L858R T790M C797S/G L792X (L792F, L792H, |
| | L792R, or L792Y) |
| 54 | EGFR L858R V834L |
| 55 | EGFR L858R T790M V834L |
| 57 | EGFR L858R T790M L792X (L792F, L792H, L792R, or L792Y) |
| 58 | EGFR L858R C797S L718Q (L718V) |
| 59 | EGFR L858R L718Q (L718V) A750P |
| 60 | EGFR L858R T790M L718Q (L718V) A750P L792V G796R |
| 61 | EGFR L861Q |
| 62 | EGFR L861Q T790M |
| 63 | EGFR L861Q T790M C797S/G/N |
| 64 | EGFR L861Q C797S/G/N |
| 65 | EGFR del18 |
| 66 | EGFR G719X (G719A, G719S, G719C, G719R, G719D, or G719V) |
| 67 | EGFR E709X (E709K, E709H, or E709A) |
| 68 | EGFR E709X (E709K, E709H, or E709A) (G719A, G719S, |
| | G719C, G719D, G719R, or G719V) |
| 69 | EGFR G719X (G719A, G719S, G719C, G719D, G719R, |
| | or G719V) S768I |
| 70 | EGFR ex20ins |
| 71 | EGFR ex20ins L718Q |
| 72 | EGFR ex20ins T790M |
| 73 | EGFR ex20ins C797S |
| 74 | EGFR S7681I |
| 75 | EGFR T790M |
| 76 | EGFR T790M C797S/G L792X (L792F, L792H, L792R, or L792Y) |

In another embodiment, NSCLC being treated with N-(2-((3 S,4R)-3-fluoro-4-methoxypiperidin-1-yl)pyrimidin-4-yl)-5-isopropyl-8-((2R,3 S)-2-methyl-3-((methyl sulfonyl)methyl)azetidin-1-yl)isoquinolin-3-amine or a pharmaceutically acceptable salt, or a pharmaceutical composition disclosed herein is characterized by EGFR comprising EGFR del19.

In another embodiment, NSCLC being treated with N-(2-((3 S,4R)-3-fluoro-4-methoxypiperidin-1-yl)pyrimidin-4-yl)-5-isopropyl-8-((2R,3 S)-2-methyl-3-((methyl sulfonyl)methyl)azetidin-1-yl)isoquinolin-3-amine or a pharmaceutically acceptable salt, or a pharmaceutical composition disclosed herein is characterized by EGFR comprising EGFR del19 T790M.

In another embodiment, NSCLC being treated with N-(2-((3 S,4R)-3-fluoro-4-methoxypiperidin-1-yl)pyrimidin-4-yl)-5-isopropyl-8-((2R,3 S)-2-methyl-3-((methyl sulfonyl)methyl)azetidin-1-yl)isoquinolin-3-amine or a pharmaceutically acceptable salt, or a pharmaceutical composition disclosed herein is characterized by EGFR comprising EGFR del19 C797S.

In another embodiment, NSCLC being treated with N-(2-((3 S,4R)-3-fluoro-4-methoxypiperidin-1-yl)pyrimidin-4-yl)-5-isopropyl-8-((2R,3 S)-2-methyl-3-((methyl sulfonyl)methyl)azetidin-1-yl)isoquinolin-3-amine or a pharmaceutically acceptable salt, or a pharmaceutical composition disclosed herein is characterized by EGFR comprising EGFR del19 C797X (C797G or C797N).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR del19 T790M C797S.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR del19 T790M C797X (C797G or C797N).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR del19 L792X (L792F, L792H or L792Y).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof, or pharmaceutical composition disclosed herein is characterized by EGFR comprising EGFR del19 T790M L792X (L792F, L792H, or L792Y).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof, or pharmaceutical composition disclosed herein is characterized by EGFR comprising EGFR del19 G796R (G796S).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof, or pharmaceutical composition disclosed herein is characterized by EGFR comprising EGFR del19 L792R (L792V or L792P).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof, or pharmaceutical composition disclosed herein is characterized by EGFR comprising EGFR del19 L718Q (L718V).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof, or pharmaceutical composition described herein is characterized by EGFR comprising EGFR del19 T790M G796R (G796S).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof, or pharmaceutical composition described herein is characterized by EGFR comprising EGFR del19 T790M L792R (L792V or L792P).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition described herein is characterized by EGFR comprising EGFR del19 T790M L718Q (L718V).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R T790M.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R C797S.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3 -((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R C797X (C797G or C797N).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R T790M C797S.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R T790M C797X (C797G or C797N).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R L792X (L792F, L792H or L792Y).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or a pharmaceutical composition disclosed herein is characterized by EGFR comprising EGFR L858R L790M L792X (L792F, L792H or L792Y).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition disclosed herein is characterized by EGFR comprising EGFR L858R G796R (G796S).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition disclosed herein is characterized by EGFR comprising EGFR L858R L792R (L792V or L792P).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition disclosed herein is characterized by EGFR comprising EGFR L858R L718Q (L718V).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition disclosed herein is characterized by EGFR comprising EGFR L858R T790M G796R (G796S).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition disclosed herein is characterized by EGFR comprising EGFR L858R T790M L792R (L792V or L792P).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, thereof or pharmaceutical composition disclosed herein is characterized by EGFR comprising EGFR L858R T790M L718Q (L718V).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR del8.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR G719X (G719A, G719S, G719C, G719R, G719D, or G719V).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR E709X (E709K, E709H, or E709A).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR E709X (E709K, E709H, or E709A) (G719A, G719S, G719C, G719D, G719R, or G719V).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR G719X (G719A, G719S, G719C, G719D, G719R, or G719V) S768I.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR ex20ins.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR ex20ins L718Q.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR ex20ins T790M.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR ex20ins C797S.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR S7681I.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR T790M.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR comprising EGFR T790M C797S/G L792X (L792F, L792H, L792R, or L792Y).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by an EGFR genotype selected from genotypes 1-76.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to osimertinib, gefitinib, erlotinib, lazertinib, aumolertinib (formerly almonertinib), olmutinib, or nazartinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to osimertinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to gefitinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to erlotinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to lazertinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to aumolertinib (formerly almonertinib).

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to olmutinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to nazartinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to afatinib, dacomitinib or avitinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3 -((methylsulfonyl)methyl)azetidin-1-yl)isoquinolin-3-amine or a pharmaceutically acceptable salt, or a pharmaceutical composition disclosed herein is characterized by EGFR mutations that confer resistance to afatinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to dacomitinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to avitinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to amivantamab.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to osimertinib and afatinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to osimertinib and dacomitinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to osimertinib and gefitinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to osimertinib and erlotinib.

In another embodiment, NSCLC being treated with N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt, or a pharmaceutical composition disclosed herein is characterized by EGFR mutations that confer resistance to amivantamab and lazertinib.

Another embodiment is the treatment a subject with metastatic NSCLC with tumors harboring activating Exon 19 Deletion or L858R EGFR mutations, G719X (A, S, C, D, R, V), S768I and L861Q, as well as a resistance mutation disclosed herein as detected by an approved molecular testing methodology.

Another embodiment is the treatment a subject with metastatic NSCLC with tumors harboring activating Exon 19 Deletion or L858R EGFR mutations as well as a resistance mutation disclosed herein as detected by an approved molecular testing methodology.

Another embodiment is a disclosed compound used in combination with a 1^{st} or 3^{rd} generation TKI indicated for the treatment of subject with metastatic NSCLC with tumors harboring T790M and C797S mutations as detected by an approved test, and whose disease has progressed on or after at least 2 prior EGFR TKI therapies.

Another embodiment is a disclosed compound used in combination with a 2^{nd} or 3^{rd} generation TKI indicated for the treatment of subject with metastatic NSCLC with tumors harboring C797X mutations as detected by an approved test, and whose disease has progressed on or after 1 or 2 prior EGFR TKI therapies.

Another embodiment is a disclosed compound for the treatment of subjects with metastatic NSCLC whose disease with on-target EGFR resistance has progressed on or after any EGFR TKI. In a specific embodiment, the disclosed compound is used in combination with a 2^{nd} or 3^{rd} generation TKI indicated for the treatment of subject with NSCLC (e.g., metastatic NSCLC).

Another embodiment is a disclosed compound for the treatment of subjects with metastatic EGFR C797X mutation-positive NSCLC as detected by an approved molecular test, whose disease has progressed on or after first-line or second-line osimertinib. In a specific embodiment, the disclosed compound is used in combination with a 2^{nd} or 3^{rd} generation TKI indicated for the treatment of subject with metastatic NSCLC.

In a particular embodiment, the deletions, mutations, and insertions disclosed herein are detected by an FDA-approved test.

A person of ordinary skill in the art can readily determine the certain EGFR alterations a subject possesses in a cell, cancer, gene, or gene product, e.g., whether a subject has one or more of the mutations or deletions described herein using a detection method selected from those known in the art such as hybridization-based methods, amplification-based methods, microarray analysis, flow cytometry analysis, DNA sequencing, next-generation sequencing (NGS), primer extension, PCR, in situ hybridization, fluorescent in situ hybridization, dot blot, and Southern blot.

To detect one or more EGFR deletions and/or mutations, a primary tumor sample, circulating tumor DNA (ctDNA), circulating tumor cells (CTC), and/or circulating exosomes may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumors samples. ctDNA is being increasingly adopted as a method to monitor response to treatment and emergenve of resistance in clinical practice. Clearance of ctDNA after 6-8 weeks of treatment is predictive of TKI benefit while increases in ctDNA or appearance of new mutations has been associated with progression (Ku B M et al. Oncology. 2022; Epub ahead of print. PMID: 35196661; Ma L. et al. Front Oncol. 2021; 11:643199; Fernandes et al. Cells. 2021; 10:1912.).

N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine, pharmaceutically acceptable salts thereof or pharmaceutical compositions disclosed herein may be used for treating to a subject who has become resistance to treatment with one or more other EGFR inhibitors. “Resistance” means that the subject's cancer previously responded to drugs but later responds poorly or not at all. In some embodiments, the subject has become refractory to one or more first generation EGFR inhibitors such as erlotinib, gefitinib, icotinib or lapatinib. In some embodiments, the subject has been become refractory to treatment with one or more second generation EGFR inhibitors such as afatinib, dacomitinib, poziotinib, or neratinib. In some embodiments the subject has become refractory to treatment with one or more first generation inhibitors and one or more second generation inhibitors. In some embodiments, the subject has become refractory to treatment with one or more third generation inhibitors such as osimertinib, nazartinib, or avitinib. In one embodiment, the subject has become refractory to treatment with one or more first generation EGFR inhibitors and one or more third generation EGFR inhibitors. In some embodiments, the subject has become refractory to treatment with one or more second generation EGFR inhibitors and one or more third generation EGFR inhibitors. In some embodiments, the subject has become refractory to treatment with one or more first generation inhibitors, and one or more third generation EGFR inhibitors.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine, or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include NaCl, lactated Ringer's, normal sucrose, normal glucose, binders, fillers, disintegrants, lubricants, coatings, sweeteners, flavors, alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Combinations

N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine, pharmaceutically acceptable salts thereof, or pharmaceutical compositions disclosed herein can be used in combination with one or more additional pharmacologically active substances. For example, the disclosure includes methods of treating a NSCLC comprising administering to a subject in need thereof N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine (or a pharmaceutically acceptable salt thereof) or a pharmaceutical composition disclosed herein thereof in combination with an EGFR (or EGFR mutant) inhibitor, such as afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)). For the treatment of cancer e.g., NSCLC using a compound of the disclosure or pharmaceutically acceptable salt thereof or pharmaceutical composition disclosed herein in combination with a first line therapy, for example a first, second, or third generation EGFR inhibitor (i.e., as an initial treatment before the cancer has become refractory) may forestall or delay the cancer from becoming refractory. Typically, the cancer is characterized by one of the EGFR genotypes described herein.

In some embodiments, in the methods disclosed herein, the subject in need thereof is co-administered with an effective amount of afatinib, dacomitinib or avitinib.

In some embodiments, in the methods disclosed herein, the subject in need thereof is co-administered with an effective amount of osimertinib, gefitinib, erlotinib, lazertinib, aumolertinib (formerly almonertinib), olmutinib, or nazartinib. In a specific embodiment, the subject in need thereof is co-administered with an effective amount (e.g., 80 mg) of osimertinib.

It will be understood that unless otherwise indicated, the administrations described herein include administering N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof prior to, concurrently with, or after administration of another pharmacologically active substance disclosed herein (e.g., osimertinib) or a pharmaceutically acceptable salt thereof described herein. In one embodiment, however, N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof is administered concurrently with another pharmacologically active substance (e.g., osimertinib) or a pharmaceutically acceptable salt thereof. In another embodiment N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof is administered sequentially with another pharmacologically active substance (e.g., osimertinib) or a pharmaceutically acceptable salt thereof. For example, N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof is administered after the administration of another pharmacologically active substance (e.g., osimertinib) or a pharmaceutically acceptable salt thereof.

### Methods of Administration and Dosage Forms

The precise amount of compound administered to provide an “effective amount” to the subject will depend on the type, and severity of the NSCLS, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day).

As used herein, the term “patient” or “subject” refers to an organism to be treated by the methods of the disclosure. Non-limiting example organisms include mammals, e.g., murines, simians, equines, bovines, porcines, canines, felines, and the like. In some embodiments, the organism is a human.

In some embodiments, the subject in need thereof is administered a total daily dose of 100 mg to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to a total daily dose of 100 mg to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered 50 mg to 250 mg twice daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 50 mg to 250 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 200 mg to 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg to 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 400 mg to 600 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 400 mg to 600 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 400 mg to 800 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 400 mg to 800 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 800 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 800 mg to 1000 mg of N-(2-((3 S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine.

In some embodiments, the subject in need thereof is administered an amount of 600 mg to 800 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 600 mg to 800 mg of N-(2-((3S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine.

In some embodiments, the subject in need thereof is administered an amount of 200 mg to 400 mg, 300 mg to 500 mg, 400 mg to 600 mg, 500 mg to 700 mg, 600 mg to 800 mg, 700 mg to 900 mg, or 800 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg to 400 mg, 300 mg to 500 mg, 400 mg to 600 mg, 500 mg to 700 mg, 600 mg to 800 mg, 700 mg to 900 mg, or 800 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 100 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 100 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg of N-(2-((3S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine.

In some embodiments, the subject in need thereof is administered an amount of 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 600 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 600 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 700 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 700 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 800 mg of N-(2-((3S,4R)-3-fluoro-4-methoxypiperidin-1-yl)pyrimidin-4-yl)-5-isopropyl-8 -((2R,3S)-2-methyl-3-((methylsulfonyl)methyl)azetidin-1-yl)isoquinolin-3-amine or a pharmaceutically acceptable salt thereof in an amount equivalent to 800 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 900 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 900 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered an amount of 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

In some embodiments, the subject in need thereof is administered N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof once daily (QD) orally with the amount identified above.

In some embodiments, the subject in need thereof is administered N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof twice daily (BID) orally with the amount identified above.

In some embodiments, the subject in need thereof is administered an amount of 50 mg of N-(2-((3 S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine or a pharmaceutically acceptable salt thereof in an amount equivalent to 50 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 100 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 100 mg of N-(2 -((3S,4R)-3-fluoro-4-methoxypiperidin-1-yl)pyrimidin-4-yl)-5-isopropyl-8-((2R,3 S)-2-methyl-3-((methylsulfonyl)methyl)azetidin-1-yl)isoquinolin-3-amine twice daily (BID).

In some embodiments, the subject in need thereof is administered an amount of 150 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 150 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 250 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 250 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 350 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 350 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 400 mg of N-(2-((3S,4R)-3-fluoro-4-methoxypiperidin-1-yl)pyrimidin-4-yl)-5-isopropyl-8 -((2R,3S)-2-methyl-3-((methylsulfonyl)methyl)azetidin-1-yl)isoquinolin-3-amine or a pharmaceutically acceptable salt thereof in an amount equivalent to 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 450 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 450 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered an amount of 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID).

In some embodiments, the subject in need thereof is administered N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount described in any one of the embodiments above (e.g., in any of the thirty-one previous paragraphs) in combination with an effective amount (e.g., 80 mg) of osimertinib. In one embodiment, the osimertinib is co-administered once daily.

In some embodiments, the subject in need thereof is administered an amount of 200 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine once daily (QD) in combination with 80 mg of osimertinib. In one embodiment, the osimertinib is co-administered once daily.

In some embodiments, the subject in need thereof is administered an amount of 300 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 300 mg of N-(2-((3S,4R)-3-fluoro-4-methoxypiperidin-1-yl)pyrimidin-4-yl)-5-isopropyl-8-((2R,3S)-2-methyl-3 -((methylsulfonyl)methyl)azetidin-1-yl)isoquinolin-3-amine once daily (QD) in combination with 80 mg of osimertinib. In one embodiment, the osimertinib is co-administered once daily.

In some embodiments, the subject in need thereof is administered an amount of 400 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine once daily (QD) in combination with 80 mg of osimertinib. In one embodiment, the osimertinib is co-administered once daily.

In some embodiments, the subject in need thereof is administered an amount of 500 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine once daily (QD) in combination with 80 mg of osimertinib. In one embodiment, the osimertinib is co-administered once daily.

In some embodiments, the subject in need thereof is administered an amount of 50 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 50 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID) in combination with 80 mg of osimertinib. In one embodiment, the osimertinib is co-administered once daily.

In some embodiments, the subject in need thereof is administered an amount of 100 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 100 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID) in combination with 80 mg of osimertinib. In one embodiment, the osimertinib is co-administered once daily.

In some embodiments, the subject in need thereof is administered an amount of 150 mg N-(2-((3S,4R)-3-fluoro-4-methoxypiperidin-1-yl)pyrimidin-4-yl)-5-isopropyl-8-((2R,3S)- 2-methyl-3-((methylsulfonyl)methyl)azetidin-1-yl)isoquinolin-3-amine or a pharmaceutically acceptable salt thereof in an amount equivalent to 150 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID) in combination with 80 mg of osimertinib. In one embodiment, the osimertinib is co-administered once daily.

In some embodiments, the subject in need thereof is administered an amount of 200 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID) in combination with 80 mg of osimertinib. In one embodiment, the osimertinib is co-administered once daily.

In some embodiments, the subject in need thereof is administered an amount of 250 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 250 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID) in combination with 80 mg of osimertinib. In one embodiment, the osimertinib is co-administered once daily.

In some embodiments, the subject in need thereof is administered an amount of 300 mg N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine twice daily (BID) in combination with 80 mg of osimertinib. In one embodiment, the osimertinib is co-administered once daily.

The pharmaceutical composition of the disclosure is formulated to be compatible with its intended route of administration. In an embodiment, the composition is formulated in accordance with routine procedures as a pharmaceutical composition adapted for oral administration to human beings.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

The following examples are intended to be illustrative and are not intended to be limiting in any way to the scope of the disclosure.

### EXEMPLIFICATION

### Example 1 A Phase 1/2 Study Targeting Acquired Resistance Mechanisms in Patients with EGFR Mutant Non-Small Cell Lung Cancer

### Part 1A: BLU-945 as Monotherapy

### Study Design

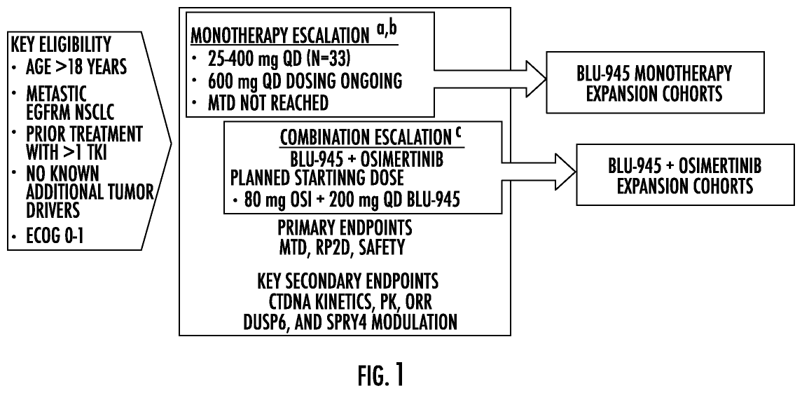

This is an ongoing Phase 1/2, open-label, first-in-human study (NCT04862780) evaluating the safety, tolerability, pharmacokinetics (PK), pharmacodynamics (PD), and anticancer activity of BLU-945 as monotherapy and in combination with osimertinib in patients with metastatic EGFRm NSCLC in the US and Japan (FIG. 1).

The study includes an initial Phase 1 portion to determine the maximum tolerated dose (MTD) and/or recommended Phase 2 dose (RP2D) of BLU-945 as monotherapy (initially in a QD (once daily) regimen with the option to evaluate BID (twice daily) dosing, if supported by emerging PK and safety data), as well as an additional dose-escalation portion to determine the RP2D of BLU-945 in combination with osimertinib.

The Phase 1 dose escalation part of the study employs a Bayesian Optimal Interval (BOIN) design with up to 12 patients evaluable for dose limiting toxicities (DLTs) for any given dose level and dose escalation will be considered complete when 12 patients are evaluable for DLT at one dose level. In the Phase 1 dose escalation, patients (aged ≥18 y; metastatic EGFRm NSCLC; ECOG PS 0-1; treated with >1 EGFR TKI) received BLU-945 mono starting at 25 mg QD. Patients progressing on osimertinib could receive BLU-945 with 80 mg osimertinib starting at 50% of the highest safe BLU-945 mono dose. Each dose escalation followed a Bayesian Optimal Interval design. Safety, including dose-limiting toxicities (DLT), PK, and circulating tumor DNA (ctDNA) mutational status were assessed.

BLU-945 was given orally in continuous 4 week cycles. The DLT evaluation period is the first 28 days (Cycle 1 of each cohort in the Phase 1 dose escalation). Patients who experience a DLT or who receive at least 75% of the prescribed BLU-945 dose (i.e., >21 days) and complete the 28-day DLT evaluation period will be evaluable for DLT assessment. Intrapatient dose escalation is permitted. Patients may continue study treatment postprogression if ongoing clinical benefit is observed (as assessed by the investigator, and approved by the Sponsor).

In addition, a twice daily (BID) dosing schedule was explored using the same BOIN dose-escalation design described for the QD dose schedule. The starting total daily dose level for the BID dose escalation will be determined by the Safety Review Committee (SRC), and the total daily dose will not exceed the highest dose level for the QD schedule that has been approved for further dose escalation by the SRC. For example, if BID dosing begins after a QD dose of 100 mg is deemed safe for further escalation, the maximum BID dose will be 50 mg BID (equivalent to a total daily dose of 100 mg).

BLU-945 and osimertinib combination treatment escalation will be initiated at 50% of the recommended phase 2 dose (RP2D) or 50% of the highest safe dose in the ongoing phase 1 BLU-945 monotherapy part of the study.

Dose level 1 will evaluate BLU-945 at 50% of the monotherapy recommended Phase 2 dose (RP2D) (or 50% of the highest dose deemed safe in Part 1A, if the monotherapy RP2D is not yet determined) in combination with full-dose osimertinib (80 mg QD). Dose escalation or de-escalation will be conducted as per the rules of the BOIN design based on observation of dose-limiting toxicity (DLT) and pharmacokinetics (PK) during the first 28-day treatment cycle as follows: Dose Level 1 (BLU-945 25% of RP2D+osimertinib 80 mg); Dose Level 2 (BLU-945 50% of RP2D+osimertinib 80 mg); Dose Level 3 (BLU-945 75% of RP2D+osimertinib 80 mg); and Dose Level 4 (BLU-945 100% of RP2D+osimertinib 80 mg). The dose level increase should be <100% of BLU-945 in the cohort(s) subsequent to cohort 1 and the next dose of BLU-945 will be selected such that the projected Area under the Curve (AUC) of BLU-945 in combination with osimertinib will not be more than the AUC of BLU-945 as monotherapy at MTD or the highest dose deemed safe in Part 1A in the combination dose-escalation. Dose escalation will not proceed beyond the BLU-945 monotherapy RP2D nor an osimertinib dose of 80 mg.

### Key Assessments (Phase 1)

The maximum tolerated dose (MTD) will be determined based on the safety profile during the first 28-day treatment cycle.

The RP2D will be determined based on DLT, PK, PD, and preliminary safety and antitumor activity data.

ctDNA will be assessed in real-time using the FoundationOne Liquid CDx assay at cycle (C) 1 day (D) 1, C1D15, and end of treatment/progression.

Response to treatment is Investigator-assessed per Response Evaluation Criteria in Solid Tumors version 1.1 every 28 days for the first 2 scans, every 8 weeks through the first year, and then every 12 weeks thereafter.

### Results

### Patients (Data Cut-Off: Mar. 9, 2022)

33 patients were treated with BLU-945 at 25-400 mg once daily (QD) in the first 5 cohorts. (Data cut-off. Mar. 9, 2022)

Baseline characteristics for these patients are shown in Table 1.

Most patients were non-smokers and the majority (n=26 [79%]) had received ≥3 lines of prior systemic therapy.

TABLE 1

| | All patients | |
|---|---|---|
| Characteristic | (N = 33) | |
| Median age (range), years | 61 | (39-78) |
| Female, n (%) | 23 | (70) |
| Race, n (%) | | |
| White | 14 | (42) |
| Asian | 18 | (55) |
| Other/unknown | 1 | (3) |
| Smoking history, n (%) | | |
| Current/former | 10 | (30) |
| Never | 22 | (67) |
| Unknown | 1 | (3) |
| ECOG PS, n (%)^{a} | | |
| 0 | 8 | (24) |
| 1 | 23 | (70) |
| 2 | 2 | (6) |
| History of Intracranial Disease, n (%) | 21 | (64) |
| Prior therapy, median (range) | 4 | (1-9) |
| Prior osimertinib, n (%) | 32 | (97) |
| 1-2 prior lines, n (%) | 7 | (21) |
| >=3 prior lines, n (%) | 26 | (79) |
| EGFR mutation status at C1D1 by | | |
| central ctDNA NGS | | |
| assessment^{b}, n (%) | | |
| EGFRm/T790M/C797S | 11 | (33) |
| EGFRm/T790M | 1 | (3.0) |
| EGFRm/C797S | 1 | (3.0) |
| EGFRm primary only | 6 | (18) |
| T790M only | 1 | (3) |
| No EGFR mutations detected | 9 | (27) |
| Not available^{c} | 4 | (12) |

C, cycle; ctDNA NGS, circulating tumor DNA next-generation sequencing; D, day; EGFRm, primary EGFR activating mutation, exon 19 deletion or L858R.
^{a}Original study protocol permitted ECOG PS of 0-2, but was later amended to ECOG PS of 0-1.
^{b}Patients with EGFR-mutant positive NSCLC were enrolled based on local mutation assessment of tumor biopsy or blood ctDNA with follow-up central ctDNA assessment at C1D1.
^{c}Results for all patients were not available at the time of the data cut.

### Safety

Safety data are summarized in Table 2. No Grade 4 or 5 adverse events (AEs) have been reported. One DLT, grade 3 transaminitis, occurred in the 400 mg QD cohort, which improved with dose interruption, and the patient remains on therapy. AEs typically associated with EGFR wildtype inhibition were minimal (rash 3%, grade 1; dry skin 3%, grade 1; diarrhea 9%, grade 1; no paronychia reported; no interstitial lung disease reported, no QTC prolongation reported). Overall, there were eight (24%) serious AEs, out of which only two (6%) were deemed to be related to the study drug: one grade 3 vomiting and one grade 3 transaminitis. No treatment discontinuations due to AEs have been reported.

TABLE 2
Most common AEs by preferred term in ≥10% of patients

| AEs, | All AEs | | Treatment-related AEs | | |
|---|---|---|---|---|---|
| regardless | N = 33 | | N = 33 | | |
| of causality, | Any | | Any | | |
| n (%) | grade | Grade 3 | grade | | Grade 3 |
| Nausea | 10 (30) | 2 (6) | 7 | (21) | 1 (3) |
| Headache | 6 (18) | 2 (6) | 1 | (3) | 0 |
| Fatigue | 6 (18) | 0 | 5 | (15) | 0 |
| Cough | 5 (15) | 0 | 1 | (3) | 0 |
| Dyspnea | 5 (15) | 1 (3) | 0 | | 0 |
| Vomiting | 5 (15) | 1 (3) | 3 | (9) | 1 (3) |
| Hyponatremia | 4 (12) | 0 | 0 | | 0 |
| Dry Mouth | 4 (12) | 0 | 3 | (9) | 0 |

### Antitumor Activity

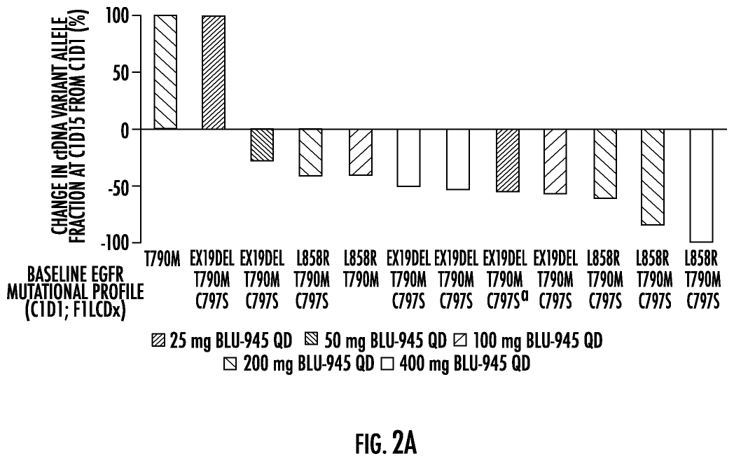

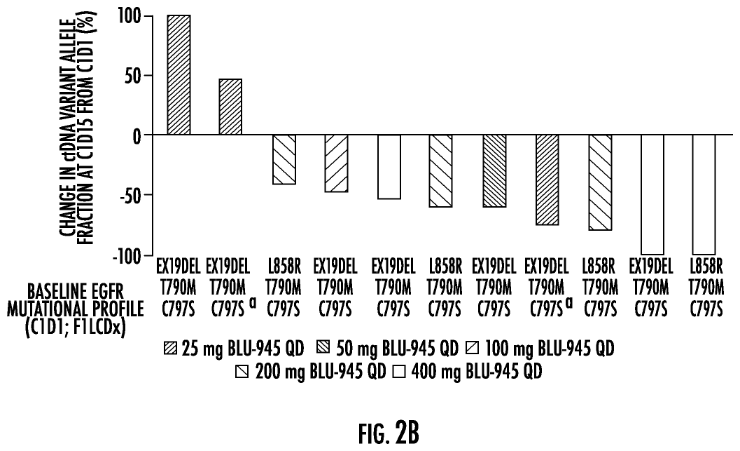

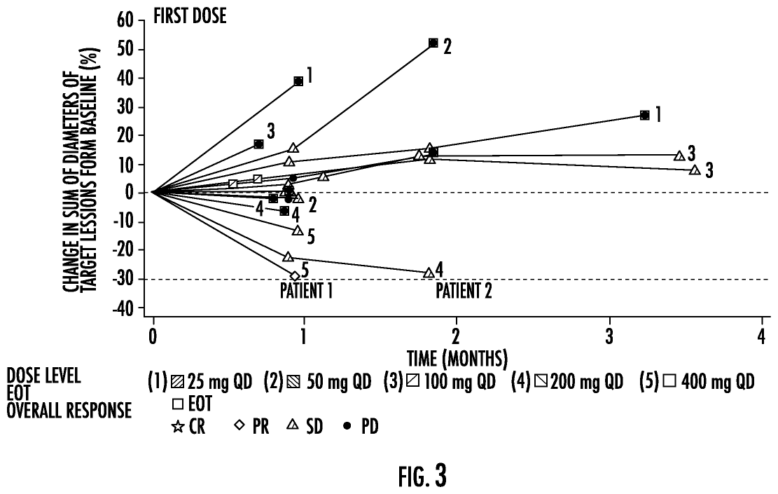

BLU-945 treatment demonstrated activity on EGFR T790M and C797S variant allele fractions as assessed by ctDNA after 14 days of therapy in every dose-cohort tested. 83% (10/12) of EGFR T790M (FIG. 2A) and 81% (9/11) of EGFR C797S (FIG. 2B) variant alleles were reduced with BLU-945 treatment. In the 400 mg cohort, all detectable T790M and C797S alleles showed reduction, including three that fell below the limit of detection (clearance). Increasing BLU-945 doses were associated with increasing anti-tumor activity (FIG. 3), with tumor shrinkage noted at doses of 200 mg QD and above, including an unconfirmed partial response at C2D1 in a patient with documented ex19del/T790M/C797S treated at 400 mg QD. Two patients started at 100 mg QD experienced stabilization of tumor growth when escalated to 200 mg at C3D1.

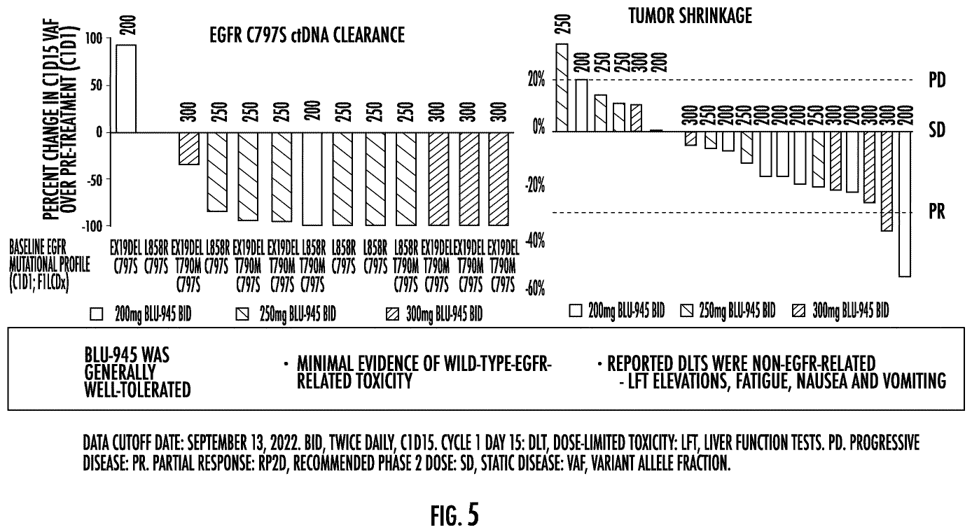

BLU-945 treatment with a twice daily (BID) dosing schedule also showed EGFR C797S ctDNA clearance and tumor shrinkage (FIG. 5).

### Patient Vignettes

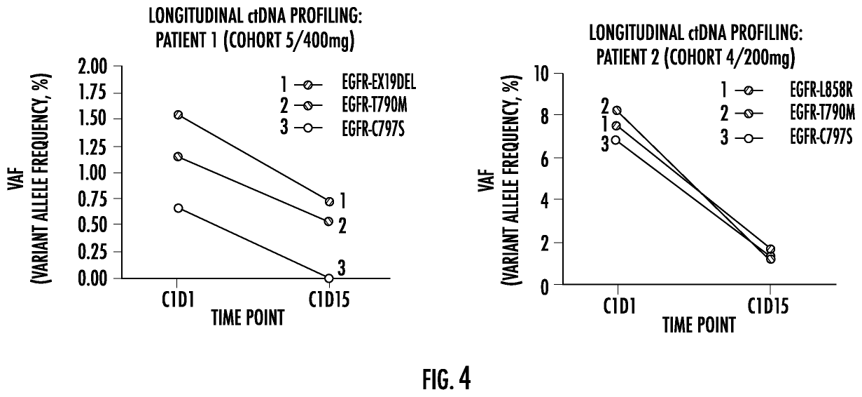

Patient 1 is a 69-year-old Caucasian female who never smoked, diagnosed with stage IV NSCLC, harboring ex19del, T790M and C797S mutations by ctDNA assessment. The patient was previously treated with platinum-based chemotherapy, erlotinib, and osimertinib, with best response of stable disease (SD) to all prior therapies. The patient had two target lesions in the liver with multiple non-target lesions in the brain, bone, liver, and retroperitoneal, and mediastinal lymph nodes. Patient 1 was started treatment at 400 mg QD and had reduction in all three detectable EGFR mutations at C1D15, with clearance of C797S and then an unconfirmed partial response with −30% tumor shrinkage at C2D1. See FIG. 4. The patient continues on therapy and tolerates treatment well.

Patient 2 is a 70-year-old Asian female who never smoked, diagnosed with EGFR L858R positive stage IVB NSCLC, harboring L858R, T790M and C797S mutations. The patient was previously treated with osimertinib and savolitinib with partial responses and had progressive disease to platinum-based chemotherapy with bevacizumab. Two target lesions in the right lung, with multiple non-target lesions in the lung and left femoral head. Patient 2 was started treatment at 200 mg QD and had reduction in all three detectable EGFR mutations at C1D15 with −21% and −28% tumor shrinkage at C2D1 and C3D1, respectively. See FIG. 4.

Conclusions for Mar. 9, 2022 data cut-off, BLU-945, a highly potent and selective oral EGFR inhibitor, was generally well tolerated at clinically active doses in heavily pre-treated patients with EGFRm NSCLC, with few adverse events (AEs) characteristic of wildtype EGFR toxicity observed at doses up to 400 mg daily.

Despite presence of EGFR mutations conferring resistance to osimertinib, treatment with BLU-945 resulted in rapid dose-dependent reduction in ctDNA, consistent with preclinical data.

Increasing BLU-945 doses were associated with increasing antitumor activity, with tumor shrinkage noted at doses of 200 mg QD and above, including an unconfirmed partial response at 400 mg.

The clonal evolution and resulting mutational complexity of EGFR-driven NSCLC tumor cells demonstrates the need for precision medicine combinations to improve clinical outcomes.

Initial safety and clinical activity results from the phase 1 SYMPHONY trial support expanded clinical development of BLU-945 in combination with osimertinib and other complementary agents.

Part 1B: BLU-945 with Osimertinib

The dose escalation for the combination of BLU-945 and osimertinib will include patients who have experienced disease progression while receiving osimertinib. During Part 1i, enrollment of patients with NSCLC harboring EGFR T790M/C797S resistance mutations is encouraged, and slots may be reserved for patients with EGFR T790M/C797S resistance mutations or other clinically relevant mutation profiles as determined by the Sponsor in discussion with the study investigators.

Dose level 1 will evaluate BLU-945 at 50% of the monotherapy RP2D (or 50% of the highest dose deemed safe in Part 1A, if the monotherapy RP2D is not yet determined) in combination with full-dose osimertinib (80 mg QD). Dose escalation or de-escalation will be conducted as per the rules of the BOIN design based on observation of DLT and PK during the first 28-day treatment cycle (Section 7.2.1.2), with a proposed escalation plan outlined in FIG. 1. The dose level increase should be <100% of BLU-945 in the cohort(s) subsequent to cohort 1 and the next dose of BLU-945 will be selected such that the projected AUC of BLU-945 in combination with osimertinib will not be more than the AUC of BLU-945 as monotherapy at MTD or the highest dose deemed safe in Part 1A in the combination dose-escalation. Dose escalation will not proceed beyond the BLU-945 monotherapy RP2D nor an osimertinib dose of 80 mg.

Patients who experience a DLT, or who receive at least 75% (i.e., >21 days) of the prescribed BLU-945 and osimertinib doses and complete the 28-day DLT evaluation period, will be evaluable for DLT assessment. The total number of patients evaluable for DLT for any given dose level should not exceed 12, and dose escalation will be considered complete when 12 patients are evaluable for DLT at one dose level. The MTD will be determined based on isotonic regression, while the RP2D, which will not exceed the MTD, may be selected with consideration to all clinical data, including safety, PK, PD, and antitumor activity.

### Part 2: Expansion Phase

### 2.1. BLU-945 Monotherapy (Groups 1, 2 and 3)

Once the BLU-945 monotherapy RP2D has been determined in Part 1A, the Phase 2 expansion will be initiated to further assess the activity, safety, PK, and PK of the BLU-945 RP2D in specific patient populations. All patients enrolled into these expansion groups will be treated with BLU-945 monotherapy at the RP2D and schedule selected in Part 1A, and will be enrolled into one of the following 3 expansion groups based on EGFR mutation profile:
Group 1 will consist of approximately 37 evaluable patients with NSCLC harboring EGFR T790M and C797S mutations, and will be conducted according to the Simon's optimal two-stage design (with 17 patients in Stage 1 and 20 patients in Stage 2).
Group 2 will include approximately 12 evaluable patients with EGFR T790M but not C797S.
Group 3 will include approximately 12 evaluable patients with EGFR C797S but not T790M.
   2.2. BLU-945 with Osimertinib (Group 4)

All patients enrolled into the BLU-945 with osimertinib expansion (Group 4) will be treated with BLU-945 and osimertinib at the RP2D and schedule selected in Part 1B. Group 4 will include approximately 24 evaluable patients, with at least 12 slots reserved for patients with EGFR T790M and C797S mutations.

BLU-945 Monotherapy (Data Cut-Off. Jan. 6, 2023)

As of 6 Jan. 2023, 108 patients have been treated with BLU-945 monotherapy in dose escalation (Part 1A) with 68 patients receiving doses ranging from 25 to 600 mg once daily (QD) and 40 patients receiving doses ranging from 100 to 300 mg twice daily (BID). As of this datacut, monotherapy dose escalation is currently ongoing with RP2D and maximum tolerated dose (MTD) yet to be determined.

### BLU-945 Monotherapy Safety

Across all monotherapy doses, 99 of 108 patients (91.7%) experienced at least one treatment emergent AE (TEAE) and 35 (32.4%) had a Grade 3 or higher AE. Only 4.6% of patients have discontinued due to a related AE. WT EGFR-related AEs were uncommon and included rash (10.2%; 0%), diarrhea (9.3%; 0%) and paronychia (1.9%; 0%). There were 12 patients with dose limiting toxicity (DLT) across the 400 mg, 500 mg, 600 mg, 200 mg BID, 250 mg BID, and 300 mg BID. Based on the available data, 600 mg QD and 300 mg BID doses were deemed not well tolerated and not explored further.

### BLU-945 Monotherapy Biomarker Efficacy

Post-osimertinib progression, many heterogenous off and on target resistance mechanisms can occur, in parallel (Leonetti A, Sharma S, Minari R, Perego P, Giovannetti E, Tiseo M. Resistance mechanisms to osimertinib in EGFR-mutated non-small cell lung cancer. Br J Cancer. 2019; 121(9):725-37. https://doi.org/10.1038/s41416-019-0573-8), which may limit the ability to assess on target activity of EGFR inhibitor by measuring simple tumor shrinkage. Therefore, ctDNA was assessed in patients at baseline (C1D1), on treatment (C1D15) and at the end of therapy.

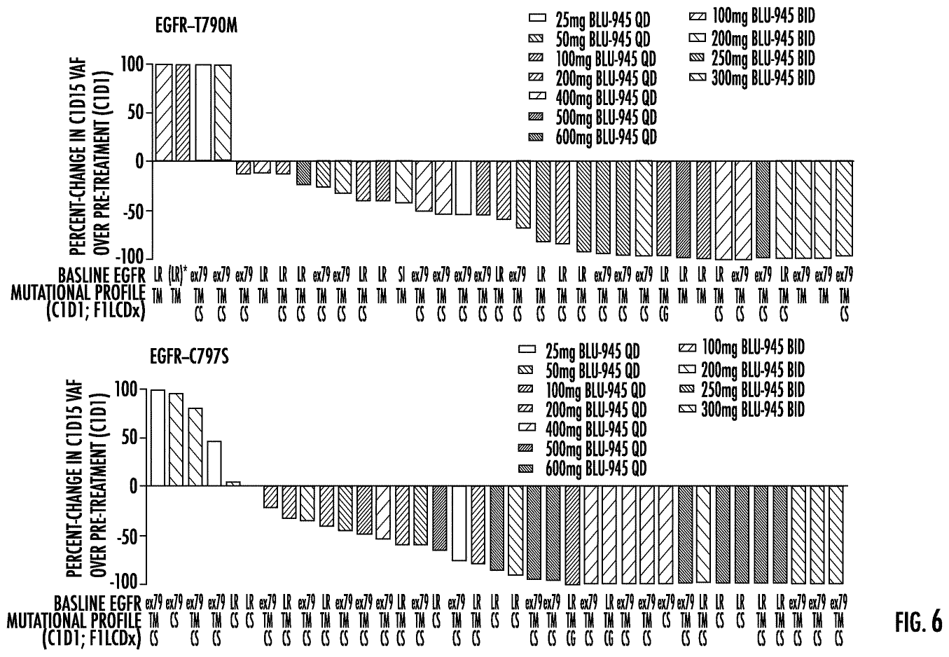

Robust dose-dependent on target activity on EGFR alleles between baseline and C1D15 were observed on the common EGFR resistance mutations T790M and C797S, including clearance of these mutations in several patients (FIG. 6).

### BLU-945 Monotherapy Clinical Activity

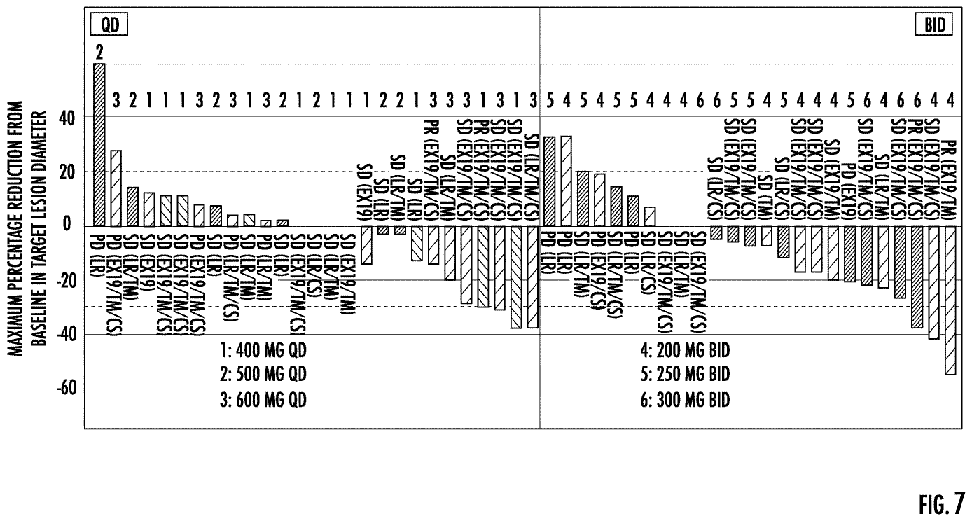

By Response Evaluation Criteria in Solid Tumors version 1.1 (RECIST 1.1), confirmed responses have occurred at doses of 400 mg QD, 200 mg BID and higher. Tumor shrinkage was observed in 10 of 27 (37%) of patients treated at >400 mg QD with one confirmed partial response (PR), and 15 of 25 (60%) of patients treated at >200 mg BID, with two confirmed PRs (FIG. 7). Four additional patients have had target lesion shrinkage of 30% or greater, but did not confirm, typically due to appearance new lesions, often in the CNS. This mixed response may be consistent with the genomic heterogeneity documented in this population at baseline, with some lesions driven to expand with non-EGFR pathways, while other lesions continue to be EGFR pathway dependent and shrink with BLU-945 therapy. A limited analysis of end of therapy ctDNA has demonstrated emergence of new drivers in some samples. A less genomically complex patient population in front line may benefit more from EGFR inhibition than in later lines.

In summary, BLU-945 monotherapy has shown promising biomarker and clinical activity in this heavily pretreated, genomically heterogeneous population, and is generally well tolerated with few discontinuations for related AEs. Importantly, BLU-945 has a differentiated safety profile from other EGFR TKIs, with minimal WT EGFR AEs, making it a good candidate to combine with other EGFR TKIs, such as the standard of care, osimertinib.

BLU-945 in Combination with Osimertinib 80 mg QD

A total of 25 patients were treated with BLU-945 (200 to 400 mg QD; 100 to 200 mg BID) in combination with 80 mg osimertinib.

BLU-945 in Combination with Osimertinib Safety

Across all combination doses, 21 out of 25 patients (84.0%) experienced at least one TEAE, including 4 (16%) with Grade 3 or higher. The frequency of WT EGFR AEs has been consistent with the addition of osimertinib, without evidence of increased WT EGFR toxicity thus far. Only one patient has discontinued due to a treated related AE thus far. The pneumonitis was a Grade 4 SAE, considered related to osimertinib, and the only DLT in combination thus far, occurring in the 300 mg QD and osimertinib 80 mg QD cohort. As of the data cut-off, there were no AEs of corrected QT interval (QTc) prolongation.

Blood samples were collected in this study for evaluation of plasma pharmacokinetics (PK) of BLU-945 and Osimertinib. Preliminary PK data comparing patients receiving BLU-945 alone to those receiving BLU-945 and Osimertinib together indicate that Osimertinib does not meaningfully affect BLU-945 PK in patients. Similarly, comparison of Osimertinib PK in combination with BLU-945 to literature reports of Osimertinib alone indicate that BLU-945 does not meaningfully affect PK of Osimertinib, suggestive of a lack of a clinically meaningful drug-drug interaction.

BLU-945 in Combination with Osimertinib Biomarker Efficacy

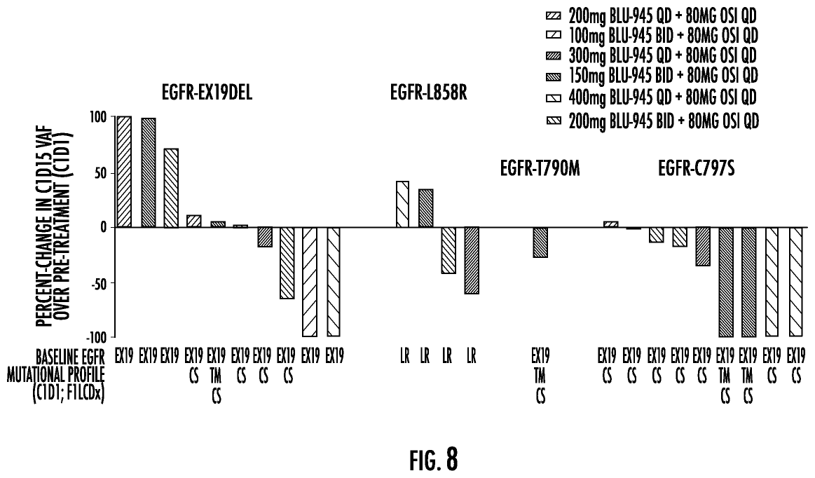

As with BLU-945 in monotherapy, the combination with osimertinib has demonstrated rapid and robust reduction and clearance of EGFR ctDNA mutant alleles, consistent with on target EGFR inhibition (see FIG. 8).

BLU-945 in Combination with Osimertinib Clinical Efficacy

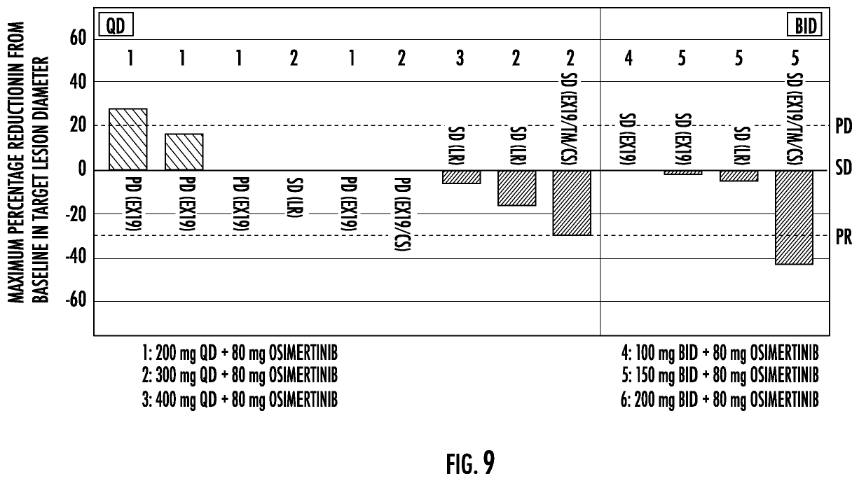

Combination dose escalation is currently exploring BLU-945 doses of 400 mg QD and 200 mg BID (which previously led to the first confirmed responses in monotherapy) with osimertinib 80 mg QD (which is the approved dose). Of note, unconfirmed responses have occurred at the 300 mg QD and 150 mg BID cohorts, with confirmatory scan data pending (see FIG. 9).

In summary, BLU-945 in combination in osimertinib has shown promising biomarker and clinical activity in patients that were progressing on osimertinib. The combination is generally well tolerated with few discontinuations for related AEs and without evidence of increased WT EGFR AE or other combination toxicity. Responses that have occurred in combination with osimertinib have occurred in patients at doses lower than first observed in BLU-945 monotherapy, which may be evidence of additive benefit of the two agents, consistent with pre-clinical data. This encouraging activity and tolerability support the expansion of this combination into the front-line setting once the combination RP2D is determined.

### BLU-945 Food Effect

BLU-945-0102 was a Phase 1, open-label, single dose, 3-way crossover study to evaluate the effect of food on the PK of BLU-945 in healthy adult subjects (N=9). 9 subjects received 100 mg BLU-945 fasted, after high-fat meal or after a snack. Overall, the results of BLU-945-0102 study supports dosing without regards to food in the Phase 2 portion of BLU-945-1101.

What is claimed is:

1. A method of treating a subject with a non-small cell lung cancer, comprising administering to the subject orally an effective amount of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine as represented by the structure below: 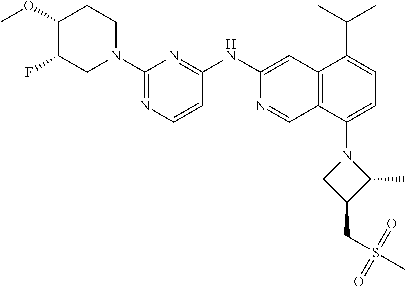
or a pharmaceutically acceptable salt thereof, and wherein the subject is co-administered with an effective amount of osimertinib.

2. The method of claim 1, wherein the subject is administered an amount of 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg to 1000 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine daily.

3. The method of claim 1, wherein the subject is administered a total daily dose of 100 mg to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to a total daily dose of 100 mg to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

4. The method of claim 1, 2 or 3, wherein the subject is administered N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof twice a day.

5. The method of claim 3, wherein the subject is administered once daily an amount of 100 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or once daily a pharmaceutically acceptable salt thereof in an amount equivalent to 100 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

6. The method of claim 3, wherein the subject is administered once daily an amount of 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or once daily a pharmaceutically acceptable salt thereof in an amount equivalent to 200 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

7. The method of claim 3, wherein the subject is administered once daily an amount of 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or once daily a pharmaceutically acceptable salt thereof in an amount equivalent to 300 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

8. The method of claim 3, wherein the subject is administered once daily an amount of 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or once daily a pharmaceutically acceptable salt thereof in an amount equivalent to 400 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

9. The method of claim 3, wherein the subject is administered once daily an amount of 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or once daily a pharmaceutically acceptable salt thereof in an amount equivalent to 500 mg of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

10. The method of claim 3, wherein the subject in need thereof is administered 50 mg to 250 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 50 mg to 250 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

11. The method of claim 3, wherein the subject in need thereof is administered 50 mg twice daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 100 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

12. The method of claim 3, wherein the subject in need thereof is administered 100 mg twice daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 100 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

13. The method of claim 3, wherein the subject in need thereof is administered 150 mg twice daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 150 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

14. The method of claim 3, wherein the subject in need thereof is administered 200 mg twice daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 200 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

15. The method of claim 3, wherein the subject in need thereof is administered 250 mg twice daily of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine or a pharmaceutically acceptable salt thereof in an amount equivalent to 250 mg twice daily (BID) of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine.

16. The method of any one of claims 1-15, wherein the non-small cell lung cancer in the subject in need thereof has metastasized.

17. The method of any one of claims 1-16, wherein the non-small cell lung cancer is characterized by epidermal growth factor receptor (EGFR) L858R mutation.

18. The method of any one of claims 1-16, wherein the non-small cell lung cancer is characterized by epidermal growth factor receptor (EGFR) exon 19 deletion.

19. The method of any one of claims 1-16, wherein the non-small cell lung cancer is characterized by EGFR L858R/T790M mutation or EGFR exon 19 deletion/T790M mutation.

20. The method of any one of claims 1-16, wherein the non-small cell lung cancer is characterized by EGFR L858R/C797X mutation or EGFR exon 19 deletion/C797X mutation.

21. The method of any one of claims 1-16, wherein the non-small cell lung cancer is characterized by EGFR L858R/T790M/C797X mutation or EGFR exon 19 deletion/T790M/C797X mutation.

22. The method of claim 20 or 21, wherein the C797X mutation is C797S mutation.

23. The method of any one of claims 1-22, wherein N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine is administered as the free base.

24. The method of any one of claims 1-23, wherein N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine and osimertinib are administered concurrently.

25. The method of any one of claims 1-23, wherein N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine and osimertinib are administered sequentially.

26. The method of any one of claims 1-25, wherein the subject in need thereof is co-administered with 80 mg of osimertinib, once a day.
